(19) United States
(12) Patent Application Publication
(10) Pub. No.: US 2006/0174363 A1
(43) Pub. Date: Aug. 3, 2006

(54) Germline transmission of avian primordial germ cells (PGCs)

(76) Inventors: Marie-Cecile Van de Lavoir, San Francisco, CA (US); Philip Albert Leighton, San Francisco, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/204,879

(22) Filed: Aug. 15, 2005

Related U.S. Application Data
(63) Continuation-in-part of application No. 11/049,229, filed on Feb. 1, 2005.

(51) Int. Cl. A01K 67/027; C12N 5/06

(52) U.S. Cl. 800/19; 435/349

(57) ABSTRACT

The present invention is long-term cultures of avian PGCs and techniques to produce germline chimeric and transgenic birds derived from prolonged PGC cultures. In some embodiments, these PGCs can be transfected with genetic constructs to modify the DNA of the PGC, specifically to introduce a transgene encoding an exogenous protein. When combined with a host avian embryo by known procedures, those modified PGCs are transmitted through the germline to yield transgenic offspring. These germline chimeric birds do not have substantial contributions of PGC-derived phenotypes in somatic cells or tissues. This invention includes compositions comprising long-term cultures of PGCs that can be genetically modified by gene targeting, that can accept large amounts of foreign DNA and that contribute to the germline of recipient embryos.

### RELATED APPLICATIONS

This application is a continuation-in-part of application Ser. No. 11/049,229 filed on Feb. 1, 2005 entitled “Long-Term Culture of Avian Primordial Germ Cells (PGCs). The priority of the prior application is expressly claimed, and the disclosure of this prior application is hereby incorporated by reference in its entirety.

This invention was made with Government support under USDA SBIR 2003-09058 and NIH IR43 GM 073306-01 and IR43 HD 047995-01. The Government has certain rights in this invention.

### BACKGROUND OF THE INVENTION

Using cell culture techniques, cells of different types can be removed from animal embryos, grown in culture, and re-introduced into live embryos. When born, the resulting animal, known as a chimera, possesses characteristics of the recipient embryo and characteristics of the donor cells grown in culture. Introducing donor cells from a culture, when the donor cells have a genotype that is distinctly different from that of the recipient embryo, can be a useful technique to study the developmental biology of an organism, or to introduce selected genetic characteristics into an organism. Furthermore, because some cells can be genetically manipulated in culture, valuable animals can be created that have characteristics that reflect the genotype of the donor cells and the genotype of the recipient embryo.

The specific characteristics of an animal created from embryo-derived donor cells introduced into a recipient embryo depend on the type of cell maintained in culture, the specific composition and characteristics of the culture conditions, the nature of the recipient embryo, and any genetic modification introduced into the cultured cells prior to introduction into the recipient embryo. The characteristics of an animal created by introducing donor cells into a recipient embryo will also differ depending on the type of tissue in the embryo to which the donor cells contribute when introduced to the embryo during development. Germline tissue includes the sperm and eggs that carry genetic information from one generation to the next. The remaining tissue, organs, bones, etc. are known as somatic tissue and do not contribute to the germline. Accordingly, different donor cell types and different culture conditions result in different contributions to the somatic tissue or germline by the donor cells as manifested in the animal born from the recipient embryo.

Considering the characteristics and classification of the cultured donor cells, embryonic stem cells or “ES cells” can be cultured and, apparently depending on the species, can be introduced into a recipient embryo and can contribute to either the somatic or germline tissue in the resulting animal. Embryonic germ cells, or “EG cells,” can also contribute to both somatic and germline tissue. Primordial germ cells, or PGCs, on the other hand, contribute exclusively to the germline to the exclusion of somatic tissue. Accordingly, when donor PGCs are grown in culture and introduced into recipient embryos, the cultured cells are present in the germline of the recipient embryo and the genetic characteristic of the cultured PGCs appear in the offspring of the animal born of the recipient embryo.

Donor ES cells have been shown to contribute to the germline of offspring of chimeras created in mice but murine PGCs have not been maintained in culture and rapidly revert to EG cells that lose the restriction to the germline. In chickens, long-term cultures of ES cells contribute to somatic tissue in chimeras, and genetic modifications introduced into the genome of ES cells in culture are exhibited in somatic chimeras. However, avian ES cells have not been shown to contribute to the germline of recipient embryos.

By inserting DNA constructs designed for tissue specific expression into ES cells in culture, chickens have been created that express valuable pharmaceutical products, such as monoclonal antibodies, in their egg whites. See PCT US03/25270 WO 04/015123 Zhu et al. A critical enabling technology for such animals is the creation and maintenance of truly long-term ES cell cultures that remain viable long enough for the genotype of the cloned cells to be engineered in culture.

Unlike ES cells, however, PGCs have only been cultured on a short-term basis. Once the length in culture extends beyond a short number of days, these cells lose the ability to contribute exclusively to the germline.

Typically, PGCs maintained in culture using current culture techniques do not proliferate and multiply. In the absence of robust growth, the cultures are “terminal” and cannot be maintained indefinitely. Over time, these terminal cell cultures are degraded and the cells lose their unique PGC morphology and revert to EG cells. Embryonic germ cells acquire a different morphology from PGCs, lose their restriction to the germline, and gain the ability to contribute to somatic tissues when injected into early stages of embryonic development. To date, long term cultures of PGCs have not been used as a vector for the introduction of foreign DNA into the genome of any organism as has been achieved with ES cells.

If the goal is to introduce a predetermined genotype into the germline of a recipient embryo, thereby enabling the animal to pass the desired genotype on to future generations, PGCs are uniquely attractive because they are known to be the progenitors of sperm and eggs. However, PGCs are notoriously difficult to grow in culture and long-term cultures of PGC cells have not been reported, but would be extremely useful if such cultures could be sustained for long periods of time.

If long-term cultures of PGCs could be created, several important advantages would result. Cultures could be created to sustain valuable genetic characteristics of important chicken breeding lines that are relied upon in the poultry and egg production industries. Currently, extraordinary measures are undertaken to prevent valuable breeding lines from being lost through accident or disease. These measures require maintaining large numbers of members of a line as breeding stock and duplicating these stocks at multiple locations throughout the world. Maintaining large numbers of valuable animals in reserve is necessary, because preserving genetic diversity within a breeding line is also important. If the valuable genetic characteristics of these birds could be preserved in PGC cell cultures, especially if the PGC cultures could be stored in liquid nitrogen, the expense of large scale reserve breeding populations could be avoided.

Long term cultures of PGCs would also be highly valuable for the production of pharmaceutical products from the eggs of genetically engineered chickens. Producing genetically engineered chickens using PGCs requires introducing genetic modifications into the genotype of the PGCs while they are maintained in culture. Techniques for a wide variety of genetic manipulations of target cells in culture are well known. However, one main difficulty is that to alter the genotype of PGCs in culture, the culture must remain viable for a length of time that is longer than the existing culture techniques allow.

Performing genetic engineering of cells in culture requires that the ideal culture conditions be maintained while genetic modifications are introduced, while cells containing the genetic modifications are selected, and while the selected cells grow and proliferate in culture. Long-term cultures of PGCs could be genetically engineered, introduced into recipient embryos, and a portion of the offspring of the recipient embryos would carry the genetic modification engineered in the cells in culture. Cells that are capable of proliferating are distinguished by their ability to generate large numbers of cells (e.g. 10⁴ to 10⁷ cells) within several days to several weeks following clonal or nearly clonal derivation. The founder cells will be the rare cells that carry the genetic modification that is desired. Typically, these cells are generated in culture at frequencies of 10⁻⁴ to 10⁻⁷ following the application of technologies for genetic modification that are well known, (e.g. lipofection or electroporation). Therefore, production of cells in culture requires passaging the cells to provide space and nutrients for the cells to proliferate and generate a sufficient number of cells to allow selection of the rare, genetically-modified cells in culture.

In addition, the culture conditions must be sufficiently robust to allow the cells to grow from an individual genetically-modified cell into a colony of 10⁴ to 10⁷ cells to be used for genetic analysis in vitro and for the production of chimeras. Thus, if the length of the culture could be extended while preserving the genotype and phenotype of the cells as true PGCs, the cells could be engineered and introduced into recipient embryos at a point in embryonic development when the germline competent cells are migrating to the gonad. These engineered PGCs would contribute exclusively to the nascent population of spermatogonia or oogonia (i.e., the sperm and eggs) in the resulting animals upon maturity. In such a resulting animal, the entirety of the somatic tissue would be derived from the recipient embryo and the germline would contain contributions from both the donor cells and the recipient embryos. Because of the mixed contribution to the germline, these animals are known as “germline chimeras.” Depending on the extent of chimerism, the offspring of germline chimeras will be derived either from the donor cell or from the recipient embryo.

Several attempts to establish long-term culture cell lines of chicken PGCs have been reported but none of these attempts has yielded a line of cells that could be sustained. In each of these cases, the culture of PGCs has differentiated into EG cells See WO 00/47717; WO99/06533; WO99/06534; Park et al., (2003) Mol Reprod Dev 65, 389-395; Park and Han, (2000) Mol Reprod Dev 56, 475-482, or cells with an ES cell phenotype, See WO 01/11019. In other cases, PGC cultures could be maintained for only 5 days (Chang et al., (1997) Cell Biology International 21, 495-499; Chang et al., (1995) Cell Biology International 19, 569-576) or 10 days (Park et al., (2003) Biol Reprod 68, 1657-1662). In another case, PGCs were maintained in culture for 2 months, but the cells proliferated only very slowly and the culture could not be passaged (Han et al., (2002) Theriogenology 58, 1531-1539). The ability of a PGC culture to be passaged is a critical property of a long-term culture used for genetic modification of PGCs and for most valuable agricultural and breeding technologies.

The ability of PGC cell cultures to proliferate is essential for selection of cells whose genome has been altered by random integration of a transgene or by site-specific modification. In both types of genetic modification, the proportion of cells acquiring the genetic modification as a stable integration into the genome of the cell in culture is very low on the order of one cell in between ten thousand and one hundred million (i.e. 1×10⁻⁴ to 1×10⁻⁸). Accordingly, the ability to establish a rapidly growing culture is required to obtain an adequate population of cells derived from the rare event that creates the genetic modification in the genome of a cell in culture.

Chicken primordial germ cells have been genetically modified using a retroviral vector within a few hours following isolation from Stage 11-15 embryos (Vick et al., (1993) Proc. R. Soc. Lond. B 251, 179-182). However, the size of the transgene is generally limited to less than about 8 kb and site-specific changes to the genome cannot be created using this technology. Stable genetic modifications requiring the insertion of greater than 15 kb of exogenous DNA into the genome of cultured avian PGCs have not been previously reported.

Any limitation on the size of an exogenous DNA transgene that may be stably introduced in a long-term PGC cell culture is a critical constraint on the ability to achieve valuable genetic modifications in the genome of PGCs in culture, and in turn, limits the types of genetic modifications that may be passed through germline to offspring of the recipient embryo. For example, the introduction of exogenous DNA encoding a protein into the genome of a transgenic chicken is a highly desirable genetic modification. If a flock of such transgenic chickens could be created, large quantities of valuable proteins could be expressed in the chicken and collected in the egg. The avian egg offers an ideal repository for biologically active proteins and provides a convenient milieu from which proteins can be isolated. Avian animals are also attractive candidates for a broad variety of transgenic technologies. However, application of the full range of mammalian transgenic techniques to avian species has been unsuccessful due to the absence of a cultured cell population into which genetic modifications can be introduced and transmitted into the germline. In a recent paper, Sang et al. state: “It is unlikely that PGCs can be maintained in culture and proliferate for the extended period necessary to identify gene targeting events without losing their ability to migrate to the developing gonad after transfer.” Prospects for Transgenesis in the Chick, Mechanisms of Development, 121, 1179-1186, (2004). Therefore, to date, genetically transfected PGCs have not been created and the transmission to a mature living animal of a genetic modification introduced into an avian PGC has not been demonstrated.

### SUMMARY OF INVENTION

This invention relates to long-term cultures of avian primordial germ cells (PGCs) and several additional inventions enabled by the creation of a long-term culture where avian PGCs proliferate and where PGC cultures can be extended through multiple passages to extend the viability of the culture beyond 40 days, 60 days, 80 days, 100 days, or longer. The PGCs of the invention proliferate in long term cultures and produce germline chimeras when injected into recipient embryos.

The PGCs maintained in the culture described herein maintain a characteristic PGC morphology while maintained in culture. The PGC morphology may be observed by direct observation, and the growth of cells in culture is assessed by common techniques to ensure that the cells proliferate in culture. Cell cultures that proliferate are defined as non-terminal and are observed to have a greater number of cells in culture at the latter of 2 distinct time points. The PGCs in the culture of the invention may have 1×10⁵ or more cells in any particular culture and this number may be observed to increase over time. Accordingly, one aspect of the invention is the observation of a proliferating PGC culture that contains a larger number of cells after a period of days, weeks, or months compared to an earlier time point in the life of the culture. Ideally, the culture contains at least 1×10⁵ cells and may be observed to have a higher number after any length of time growing in culture. Furthermore, the PGCs may be observed to be the dominant species in the culture such that, when considering the minimal contribution made by non-chicken feeder cells, the proliferating component of the cell culture consists essentially of chicken primordial germ cells, to the substantial exclusion of other chicken-derived cells.

The culture also manifests the characteristic of allowing proliferation by passage such that samples or aliquots of cells from an existing culture can be separated and will exhibit robust growth when placed in new culture media. By definition, the ability to passage a cell culture indicates that the cell culture is growing and proliferating and is non-terminal. Furthermore, the cells of the invention demonstrate the ability to create germline chimeras after several passages and maintain a PGC morphology. As described herein, this proliferation is an essential feature of any cell culture suitable for stable integration of exogenous DNA sequences.

The PGCs of the invention can be obtained by any known technique and grows in the culture conditions described herein. However, it is preferred that whole blood is removed from a stage 15 embryo and is placed directly in the culture media described below. This approach differs from other approaches described in the literature wherein PGCs are subjected to processing and separation steps prior to being placed in culture. Unlike conventional culture techniques, the culture and methodology of the present invention relies on robust differential growth between PGCs and other cells from whole blood that may initially coexist in the medium, in order to provide the large populations of PGCs in culture described here. Accordingly, the present invention provides culture of PGCs derived directly from whole blood that grow into large cell concentrations in culture, can go through an unlimited number of passages, and exhibit robust growth and proliferation such that the PGCs in culture are essentially the only cells growing and proliferating. These culture conditions provide an important advantage of the present invention, thereby rendering the collection, storage, and maintenance of PGCs in culture particularly simple and efficient and providing a readily available source of donor cells to create germline chimeras that pass the genotype of cultured PGC cells to offspring.

The PGCs maintained in culture by the inventors have demonstrated the existence of a non-terminal culture and have currently existed for at least 327 days in culture. These cells are growing and proliferating in the same manner as was observed at 40, 60, 80, or 100 days (and all integral values therein) and the cells continue to contribute to germline chimeras as described below, and thus, exhibit the primary distinguishing characteristics of true PGCs, i.e., the exclusive contribution to the germline when introduced into a recipient embryo. The culture methodology of the invention includes using whole blood, which contains red blood cells and other metabolically active cell types, placing a mixture of cells into culture along with primordial germ cells and allowing the culture to evolve to consist essentially of avian PGCs displaying the long-term culture characteristics described herein. Cell culturing technology described herein avoids any cell separation processes or techniques and relies solely on differential growth conditions to yield the predominance of PGCs in culture. The use of whole blood as the source of the established and cultured PGC cells offers practical advantages in the efficiency and utility of establishing the cultures and using the cells for agricultural or transgenic purposes. Accordingly, in one aspect of the invention, the culture medium is conditioned with BRL (Buffalo Rat Liver cells), contains fibroblast growth factors, stem cell factor, and chicken serum. The particular characteristics of the medium are described in greater detail below.

In one aspect of the present invention, a culture is established that has a large number of PGCs that are genetically identical and which proliferate to yield a long-term cell culture. These PGCs can be used repeatedly to create germline chimeras by introducing the PGCs from a proliferating long-term culture to recipient embryos. In previous attempts to use PGCs to create germline chimeras, the number of chimeras that could be created was inherently limited by the inability to grow long-term cultures of true PGCs that retain the PGC phenotype. Because long-term cultures are enabled by the present invention, any number of germline chimeras can be created from the same cell culture and an entire population of germline chimeras can be established having genetically identical, PGC-derived germlines. Accordingly, one aspect of the present invention is the creation of large numbers, including greater than 3, greater than 4, greater than 5, 10, 15 and 20 germline chimeric animals all having genetically identical PGC-derived cells in their germline. Another aspect of the invention is the creation of a population of germline chimeras having genetically identical PGC-derived cells in their germline that have, within the population, age differentials that reflect the use of the same long-term cell culture to create germline chimeras. The age differentials exceed the currently available ability to culture primordial germ cells over time and are as high as 190 days without freezing. Accordingly, the present invention includes two or more germline chimeras having identical PGC-derived cells in their germline that differ in age by more than 40 days, 60 days, 80 days, 100 days, 190 days, etc., or any other integral value therein—without freezing the cells. The invention also includes the existence of sexually mature germline chimeras having genetically identical PGC-derived cells in their germline, together with the existence of a non-terminal PGC culture used to create these germline chimeras and from which additional germline chimeras can be created.

Because the PGCs can be maintained in culture in a manner that is extremely stable, the cells can also be cryo-preserved and thawed to create a long-term storage methodology for creating germline chimeras having a capability to produce offspring defined by the phenotype of the PGCs maintained in culture.

The capability to produce large numbers of germline chimeras also provides the ability to pass the PGC-derived genotype through to offspring of the germline chimera. Accordingly, the present invention includes both populations of germline chimeras having genetically identical PGC-derived cells in the germline, but also offspring of the germline chimeras whose genotype and phenotype is entirely determined by the genotype of the PGCs grown in culture. Transmission of a PGC-derived phenotype through the germline has been observed for more than 20 birds at transmission percentages as high as 86%. Thus, the invention includes the offspring of a germline chimera created by germline transmission of a genotype of a primordial germ cell held in culture. Accordingly, the invention includes each of the existence of a primordial germ cell culture containing PGCs of a defined phenotype, a germline chimera having the same primordial germ cells as part of its germline, and an offspring of the germline chimera having a genotype and phenotype dictated by the PGCs in culture.

As has been described previously, the existence of long-term PGC cultures enables the ability to stably transfect the cells in culture with DNA encoding exogenous proteins or introducing other desirable genetic manipulations such as gene insertions and knock-outs of a transgenic animal. Accordingly, all of the above-described populations of PGCs in culture, germline chimeras, and offspring of germline chimeras can also be comprised of a DNA construct stably integrated into the genome of the primordial germ cell, transmitted into the germline of the germline chimera, and transmitted into future generations comprised of offspring of the germline chimeras.

The primordial germ cells may contain virtually any engineered genetic constructs and may be used to introduce genetic modifications into birds that exceed the size restrictions currently imposed by retroviral technologies, including the site-specific insertion of transgenes encoding full length exogenous proteins such as monoclonal antibodies. In a preferred embodiment, genetically engineered chickens express exogenous proteins in a tissue specific fashion in the oviduct to express exogenous proteins in the egg.

The PGC cultures of the invention are sufficiently stable to allow a transgene to become stably integrated into the genome of the PGC, to isolate the genetically modified cells from non-modified cells in the culture, and to introduce the modified cells into a recipient embryo, while maintaining the ability of the cultured PGCs to contribute to the germline in a resulting chimera. In cases where expression of the transgene is controlled by a tissue specific promoter, the transgene would not be expressed in PGCs. In these cases, the transgene would be expressed in the selected tissues in transgenic offspring of the germline chimera. Whole genomes can be transferred by cell hybridization, intact chromosomes by microcells, subchromosomal segments by chromosome mediated gene transfer and DNA fragments in the kilobase range by DNA mediated gene transfer (Klobutcher, L. A. and F. H. Ruddle, Ann. Rev. Biochem., 50: 533-554, 1981). Intact chromosomes may be transferred by microcell-mediated chromosome transfer (MMCT) (Fournier, R. E. and Ruddle, F. H., Proc. Natl. Acad. Sci., USA 74: 319-323, 1977).

Stable long-term cultures of PGCs that yield genetically engineered chickens are necessary for several applications in avian transgenesis, including the production of proteins for the pharmaceutical industry, production of chickens that deposit human monoclonal antibodies in their eggs, and to make site-specific changes to the avian genome for any number of other applications including human sequence polyclonal antibodies.

The ratio of donor-derived and recipient-derived PGCs in a recipient embryo can be altered to favor colonization of the germline in PGC-derived chimeras. In developing chicken and quail embryos, exposure to busulfan either greatly reduces or eliminates the population of primordial germ cells as they migrate from the germinal crescent to the gonadal ridge (Reynaud (1977a) Bull Soc Zool Francaise 102, 417-429; Reynaud (1981) Arch Anat Micro Morph Exp 70, 251-258; Aige-Gil and Simkiss (1991) Res Vet Sci 50, 139-144). When busulfan is injected into the yolk after 24 to 30 hours of incubation and primordial germ cells are re-introduced into the vasculature after 50 to 55 hours of incubation, the germline is repopulated with donor-derived primordial germ cells and subsequently, donor derived gametes are produced (Vick et al. (1993) J Reprod Fert 98, 637-641; Bresler et al. (1994) Brit Poultry Sci 35 241-247).

Methods of the invention include: obtaining PGCs from a chicken, such as from the whole blood of a stage 15 embryo, placing the PGCs in culture, proliferating the PGCs to increase their number and enabling a number of passages, creating germline chimeras from these long-term cell cultures, and obtaining offspring of the germline chimeras having a genotype provided by the cultured PGCs. The methods of the invention also include inserting genetic modifications into a population of PGCs in culture to create stably transfected PGCs, selecting cells from this population that carry stably integrated transgenes, injecting the genetically modified cells carrying the stably integrated transgenes into a recipient embryo, developing the embryo into a germline chimera containing the genetic modification in the germline, raising the germline chimera to sexual maturity and breeding the germline chimera to obtain genetically modified offspring wherein the genetic modification is derived from the cultured PGC.

### DESCRIPTION OF THE FIGURES

FIG. 1A: PGCs maintained in culture for 54 days. Note that the cells are not attached and maintain a round morphology. Arrows indicate several dividing cells visible in this culture.

FIG. 1B: PGCs maintained in culture for 234 days. These cells are cultured on a feeder layer of irradiated STO cells.

FIG. 2: Gene expression as determined by RT-PCR of the germ cell markers CVH and Dazl. Cells were in culture for 32 days. Lane1 shows expression of both CVH and Dazl in an aliquot of PGCs. A second sample, in lane 2, did not have sufficient mRNA as determined by the absence of actin. CES cells were also analyzed; actin was expressed but the cES cells did not express CVH and Dazl was expressed only weakly.

FIG. 3: Western analysis of PGCs maintained in culture for 166 days. Testis was used as positive control and liver as a negative control. Rabbit anti-chicken CVH IgG was used as the primary antibody.

FIG. 4: Telomeric Repeat Amplification Protocol (TRAP) Assay. Different dilutions of cell extracts of 2 different PGC cell lines (13&16) maintained in culture for 146 days. The positive control consisted of the transformed human kidney cell line 293 and the negative control was lysis buffer only with no template added. In the PGC and positive control lanes, repeat sequences are visible indicating the presence of telomerase.

FIG. 5A: cEG cells derived from PGCs maintained in culture.

FIG. 5B: Chicken embryonic stem cells. Note the small cells, big nucleus (light grey) and pronounced nucleolus in both cell types.

FIG. 6: Chimeras obtained from cEG cells derived from PGCs. The EG cells were derived from black feathered Barred Rock embryos. As recipients, a white feathered (White Leghorn) embryo was used. Somatic chimerism is evident by the black feathers.

FIG. 7. Rooster IV7-5 with his offspring. A White Leghorn is homozygous dominant at the dominant, white locus (I/I). When bred to a Barred Rock hen (i/i) all offspring from a White Leghorn will be white (I/i). A black chick demonstrates that the injected PGCs (derived from a Barred Rock embryo (i/i)) have entered the germline of the White Leghorn rooster.

FIG. 8. Southern analysis of cx-neo transgene in a line of primordial germ cells (PGCs).

FIG. 9: FACS analysis of cells stained with antibodies against chicken vasa homologue (CVH) and 1B3. The cell lines used were PGC 102; ES 439 and EG 455.

FIG. 10: Southern analysis of the HS4-β-actin-neo transgene in 2 lines of primordial germ cell PGCs.

FIG. 11: Southern analysis of the HS4β-actin-eGFP-β-actin-puro transgene in primordial germ cell (PGC) line TP103.

FIG. 12: Karyotype of G-09 showing all chromosomes to be diploid. In one copy of GGA 2, the majority of the p arm is either missing or translocated to another chromosome. The other copy of CGA 2 is normal. The cells are ZZ (male).

FIG. 13: Section of testes, at 18 days of development, stained with DAPI. GFP positive germ cells are clearly visibly within the seminiferous tubules.

FIG. 14: The DAPI stained panel shows a section through a seminiferous tubule of an E18 testis.

### DETAILED DESCRIPTION OF INVENTION

As used herein, the terms chicken embryonic stem (cES) cells mean cells exhibiting an ES cell morphology and which contribute to somatic tissue in a recipient embryo derived from the area pellucida of Stage X (E-G&K) embryos (the approximate equivalent of the mouse blastocyst). CES cells share several in vitro characteristics of mouse ES cells such as being SSEA-1⁺, EMA-1⁺ and telomerase⁺. ES cells have the capacity to colonize all of the somatic tissues.

As used herein, the terms primordial germ cells (PGCs) mean cells exhibiting a PGC morphology and which contribute exclusively to the germline in recipient embryos, PGCs may be derived from whole blood taken from Stage 12-17 (H&H) embryos. A PGC phenotype may be established by (1) the germline specific genes CVH and Dazl are strongly transcribed in this cell line, (2) the cells strongly express the CVH protein, (3) the cells do not contribute to somatic tissues when injected into a Stage X nor a Stage 12-17 (H&H) recipient embryo, (4) the cells give rise to EG cells (see below), or (5) the cells transmit the PGC genotype through the germline when injected into Stage 12-17 (H&H) embryos (Tajima et al. (1993) Theriogenology 40, 509-519; Naito et al., (1994) Mol Reprod Dev, 39, 153-161; Naito et al., (1999) J Reprod Fert 117, 291-298).

As used herein, the term chicken embryonic germ (cEG) cells means cells derived from PGCs and are analogous in function to murine EG cells. The morphology of cEG cells is similar to that of cES cells and cEG cells contribute to somatic tissues when injected into a Stage X (E-G&K) recipient.

The germline in chickens is initiated as cells from the epiblast of a Stage X (E-G & K) embryo ingress into the nascent hypoblast (Kagami et al., (1997) Mol Reprod Dev 48, 501-510; Petitte, (2002) J Poultry Sci 39, 205-228). As the hypoblast progresses anteriorly, the pre-primordial germ cells are swept forward into the germinal crescent where they can be identified as large glycogen laden cells. The earliest identification of cells in the germline by these morphological criteria is approximately 8 hours after the beginning of incubation (Stage 4 using the staging system established by Hamburger and Hamilton, (1951) J Morph 88, 49-92). The primordial germ cells reside in the germinal crescent from Stage 4 (H&H) until they migrate through the vasculature during Stage 12-17 (H&H). At this time, the primordial germ cells are a small population of about 200 cells. From the vasculature, the primordial germ cells migrate into the genital ridge and are incorporated into the ovary or testes as the gonad differentiates (Swift, (1914) Am J Anat 15, 483-516; Meyer, (1964) Dev Biol 10,154-190; Fujimoto et al. (1976) Anat Rec 185,139-154). In all species that have been examined to date, primordial germ cells have not proliferated in culture for long periods without differentiating into EG cells. Long periods in culture are required in order to produce a sufficient number of cells to introduce a genetic modification by conventional electroporation or lipofection protocols. Typically, these protocols require 10⁵ to 10⁷ cells and therefore, production of these cells from a single precursor requires 17 to 24 doublings assuming that all cell divisions are (1) synchronous and (2) produce two viable daughter cells. The introduction of a genetic modification into the genome of a cell is a rare event, typically occurring in one in 1×10⁴ to 1×10⁶ cells. Following genetic modification, the cells must be able to establish a colony from the single cell that carries and/or expresses the genetic modification. The colony must be able to expand into a population of 10⁵ to 10⁷ cells that can be analyzed by PCR or Southern analysis to evaluate the fidelity of the transgene and provide a sufficient number of cells that are then injected into recipient Stage 13-15 (H&H) embryos. Therefore another 17 to 24 cell divisions are required to produce the populations of cells and in total 34 to 58 doublings are required to produce the population of genetically modified cells. Assuming that the cell cycle is 24 hours, a minimum of 34 days and in general 58 days in culture are required to produce genetically modified primordial germ cells for injection into Stage 13-15 (H&H) recipient embryos. The injected cells must then be able to colonize the germline, form functional gametes and develop into a new individual post fertilization.

The following describes the unexpected finding that PGCs can be isolated from the blood of Stage 12-17 (H&H) embryos, that the cells will proliferate rapidly and maintain their PGC phenotype, that the PGCs can be passaged at daily or 2-day or 3-dayintervals, that the PGCs will colonize the germline but not somatic tissues after at least 110 days in culture, that viable offspring can be obtained from cells that have been in culture for 110 days, that the PGCs can be genetically modified by transfection with a transgene, and that the genetically modified PGCs can be isolated and propagated into a colony of genetically modified PGCs.

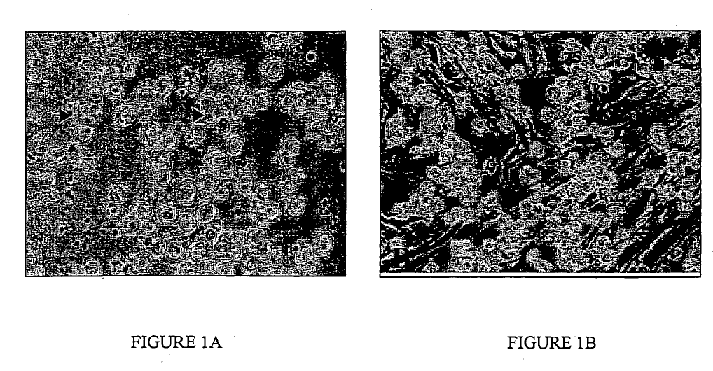

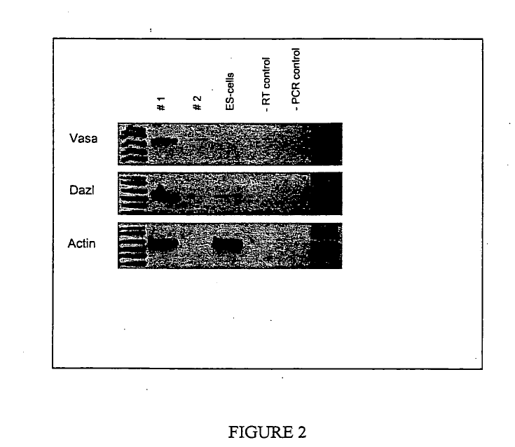

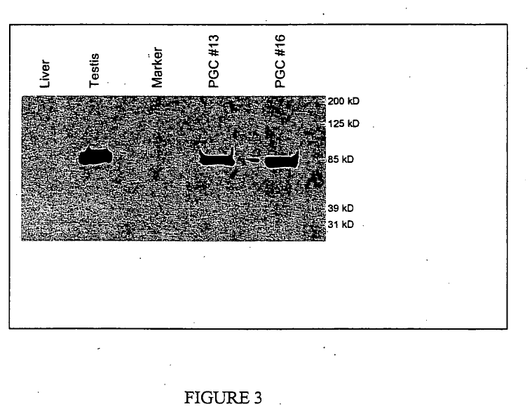

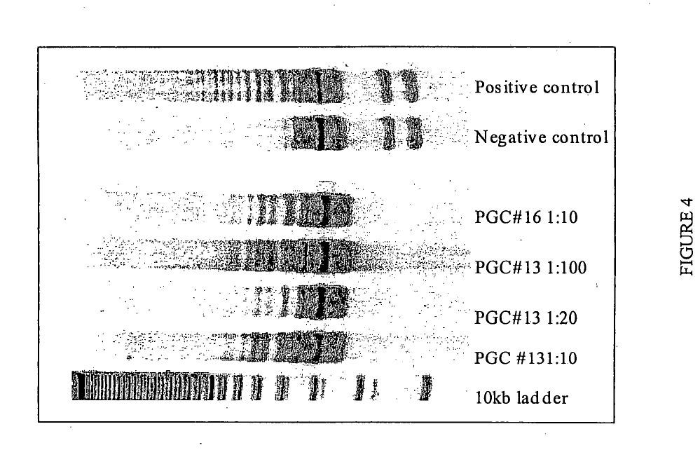

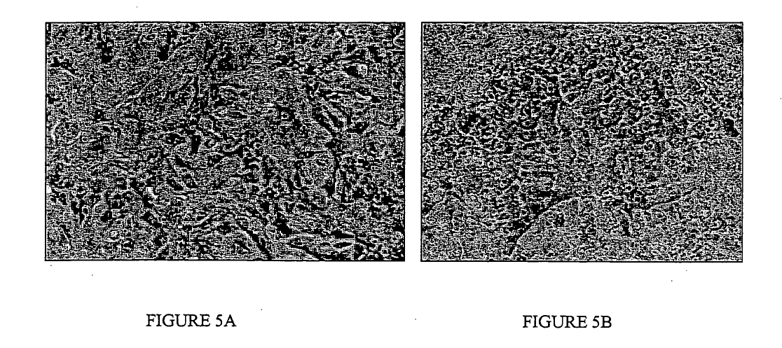

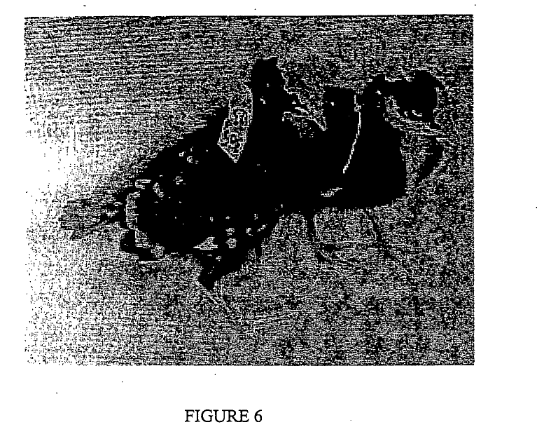

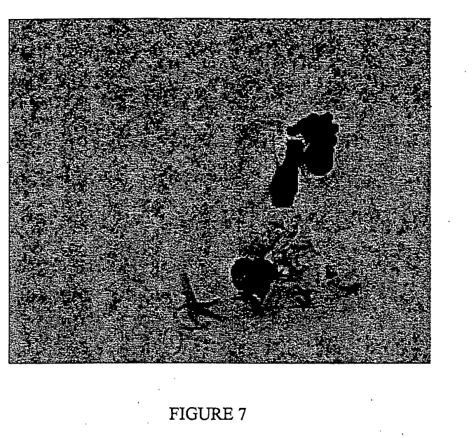

Pursuant to this invention, chicken PGC cell lines have been derived from blood taken from Stage 14-16 (H&H) embryos that have a large, round morphology (FIG. 1). These cells are confirmed to be chicken PGCs by their morphology after long term culturing and their ability to yield PGC-derived offspring. In addition, the PGC cultures express the germline-specific genes Dazl and CVH (FIG. 2) and the CVH protein is produced by the cells in culture (FIG. 3). PGCs in culture also express telomerase (FIG. 4) indicating that they have an immortal phenotype. Furthermore, PGCs will give rise to embryonic germ (EG) cells in the appropriate culture conditions (FIG. 5). By analogy, mouse and human PGCs will give rise to EG cells when treated in an analogous fashion. Mouse EG cells will contribute to somatic tissues and chicken EG cells also contribute to somatic tissues as indicated by black feather pigmentation in chimeras (FIG. 6). Chicken PGCs have been genetically modified as indicated by Southern analysis (FIG. 7). In a preferred embodiment, the CX promoter is stably integrated into the genome of a PGC and is used to facilitate expression of the gene encoding aminoglycoside phosphotransferase (APH) which is also integrated into the genome of a PGC and is used to confer resistance to neomycin added to culture media in order to select PGCs that have been genetically modified.

### EXAMPLE 1

### Derivation of Cultures of Chicken PGCs

Two to five μL of blood taken from the sinus terminalis of Stage 14-17 (H&H) embryos were incubated in 96 well plates in a medium containing Stem Cell Factor (SCF; 6 ng/ml or 60 ng/ml), human recombinant Fibroblast Growth Factor (hrFGF; 4 ng/ml or 40 ng/ml), 10% fetal bovine serum, and 80% KO-DMEM conditioned medium. The wells of the 96-well plates was seeded with irradiated STO cells at a concentration of 3×10⁴ cells/cm².

KO-DMEM conditioned media were prepared by growing BRL cells to confluency in DMEM supplemented with 10% fetal bovine serum, 1% pen/strep; 2 mM glutamine, 1 mM pyruvate, 1× nucleosides, 1× non-essential amino acids and 0.1 mM β-mercaptoethanol and containing 5% fetal bovine serum for three days. After 24 h, the medium was removed and a new batch of medium was conditioned for three days. This was repeated a third time and the three batches were combined to make the PGC culture medium.

After approximately 180 days in culture, one line of PGCs was grown in media comprised of 40% KO-DMEM conditioned media, 7.5% fetal bovine serum and 2.5% chicken serum. Under these conditions, the doubling time of the PGCs was approximately 24-36 hours.

When the culture was initiated, the predominant cell type was fetal red blood cells. Within three weeks, the predominant cell type was that of a PGC. Two PGC cell lines were derived from 18 cultures that were initiated from individual embryos.

A line of PGCs has been in culture for over 9 months, maintain a round morphology, and remain unattached (FIGS. 1A &B). PGCs have been successfully thawed after cryopreservation in CO₂ independent medium containing 10% serum and 10% DMSO.

### EXAMPLE 2

### Cultured PGCs Express CVH and Dazl

Expression of CVH, which is the chicken homologue of the germline specific gene VASA in Drosophila, is restricted to cells within the germline of chickens and is expressed by approximately 200 cells in the germinal crescent (Tsunekawa et al., 2000). CVH expression is required for proper function of the germline in males; loss of CVH function causes infertility in male mice (Tanaka et al., 2000). The expression of Dazl is restricted to the germline in frogs (Houston and King, 2000) axolotl (Johnson et al., 2001), mice (Schrans-Stassen et al., 2001), rat (Hamra et al., 2002), and human (Lifschitz-Mercer et al., 2000). Deletion of Dazl led to spermatogenic defects in transgenic mice (Reijo et al., 1995).

After 32 days, PGCs were washed with PBS, pelleted and mRNA was isolated from the tissue samples with the Oligotex Direct mRNA kit (Qiagen). cDNA was then synthesized from 9 μl of mRNA using the SuperScript RT-PCR System for First-Strand cDNA synthesis (Invitrogen). Two μl of cDNA was used in the subsequent PCR reaction. Primer sequences which were derived from the CVH sequence (accession number AB004836), Dazl sequence (accession number AY211387), or β-actin sequence (accession number NM_{—}205518) were

| | | |
|---|---|---|
| V-1 | GCTCGATATGGGTTTTGGAT | (SEQ ID NO. 1) |
| V-2 | TTCTCTTGGGTTCCATTCTGC | (SEQ ID NO. 2) |
| Dazl-1 | GCTTGCATGCTTTTCCTGCT | (SEQ ID NO. 3) |
| Dazl-2 | TGC GTC ACA AAG TTA GGC A | (SEQ ID NO. 4) |
| Act-RT-1 | AAC ACC CCA GCC ATG TAT GTA | (SEQ ID NO. 5) |
| Act-RT-2 | TTT CAT TGT GCT AGG TGC CA | (SEQ ID NO. 6) |

Primers V-1 and V-2 were used to amplify a 751 bp fragment from the CVH transcript. Primers Dazl-1 and Dazl-2 were used to amplify a 536 bp fragment from the Dazl transcript. Primers Act-RT-1 and Act-RT-R were used to amplify a 597 bp fragment from the endogenous chicken β-actin transcript. PCR reactions were performed with AmpliTaq Gold (Applied Biosystems) following the manufacturer's instructions.

### EXAMPLE 3

### PGCs Express the CVH Protein

Protein was extracted from freshly isolated PGCs using the T-Per tissue protein extraction kit (Pierce). Protein from cells was extracted by lysing the cells in 1% NP₄O; 0.4% deoxycholated 66 mM EDTA; 10 mM, Tris, pH7.4. Samples were run on 4-15% Tris-HCL ready gel (Bio-Rad). After transfer onto a membrane, Western blots were performed with Super Signal West Pico Chemiluminescent Substrate kits (Pierce) as instructed. A rabbit anti-CVH antibody was used as a primary antibody (1:300 dilution) and a HRP-conjugated goat anti-rabbit IgG antibody (Pierce, 1:100,000) was used as a secondary antibody (FIG. 3).

### EXAMPLE 4

### Cultured PGCs Express Telomerase

Primordial germ cells were pelleted and washed with PBS before being frozen at −80° C. until analysis. Cell extracts were prepared and analyzed according to the manufacturer's directions using the TRAPeze Telomerase Detection Kit (Serologicals Corporation) which is based upon the Telomeric Repeat Amplification Protocol (TRAP) (Kim et al., 1994). FIG. 4.

### EXAMPLE 5

### Embryonic Germ (EG) Cells Can be Derived from Cultures of PGCs

Chicken EG cells have been derived from PGCs by allowing the cells to attach to the plate and subsequently removing FGF, SCF and chicken serum; these conditions are the same as those for ES cell culture. The morphology of the cEG cells is very similar to the cES cells (FIG. 5A,B). When cEG cells are injected into Stage X (E-G&K) embryos, they have the ability to colonize somatic tissues and make chimeras that, as juveniles, appear identical to chimeras made with cES cells (FIG. 6).

### EXAMPLE 6

### Cultured PGCs Give Rise to Functional Gametes

PGCs that were cultured for 40 days or 110 days were injected into Stage 13-16 (H&H) White Leghorn embryos and 23 chicks have hatched. All of these chicks are phenotypically White Leghorns. The males were reared to sexual maturity and have been mated to Barred Rock hens (Table 1). The rate of germline transmission of the roosters varied from <1% to 87% (Table 1 and FIG. 7).

TABLE 1
The rate of germline transmission of roosters, injected
with PGCs that had been cultured for 40 and 110 days.

| | Days in | # | # Black | % Germline |
|---|---|---|---|---|
| Rooster | culture | Offspring | Offspring | Transmission |
| IV7-5 | 40 | 460 | 95 | 17 |
| IV7-6 | 40 | 672 | 1 | 0.1 |
| IV7-22 | 40 | 306 | 5 | 2 |
| IV9-1 | 110 | 470 | 410 | 87 |
| IV9-2 | 110 | 584 | 19 | 3 |
| IV9-13 | 110 | 341 | 2 | 0.6 |
| IV9-15 | 110 | 482 | 7 | 1.5 |
| IV9-48 | 110 | 356 | 5 | 1.4 |

PGCs may also be injected into the subgerminal cavity of stage X embryos. 1000 or 5000 PGCs were injected after 209 days of culture into irradiated embryos. Hatched male chicks were grown to sexual maturity and bred to test for germline transmission. In 3 out of 4 roosters tested germline transmission observed in varying frequency of 0.15 to 0.45%. This indicates that PGCs can colonize the germline when injected before gastrulation.

### EXAMPLE 7

### Sensitivity of PGCs to Neomycin and Puromycin

The sensitivity of PGCs to puromycin and neomycin was determined to establish the concentration of puromycin and neomycin required to allow the growth of cells that express antibiotic resistance under the control of the CX-promoter which is strongly expressed in all tissues (Origen Therapeutics, unpublished). These experiments demonstrated that a concentration of 300 μg/ml neomycin for 10 days is necessary to eliminate all non-transfected cells. A concentration of 0.5 μg/ml puromycin was sufficient to eliminate PGCs within 7-10 days.

### EXAMPLE 8

### Genetic Modification of PGCs

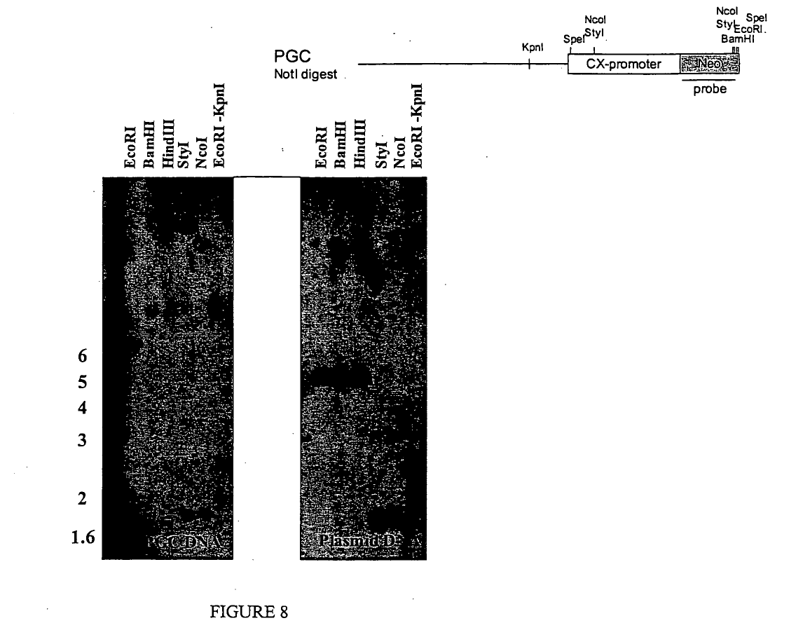

Twenty microgram (20 μl) of a NotI linearized cx-neo transgene (see FIG. 8) was added to a population of 5.8×10⁶ PGCs that had been in culture for 167 days. The cells and DNA were resuspended in 800 μl of electroporation buffer and 8 square wave pulses of 672 volts and 100 μsec duration were applied. After ten minutes, the cells were resuspended in culture medium and aliquoted into 24-well plates. Two days after electroporation, 300 μg of neomycin were added per ml of medium to select cells that were expressing the cx-neo transgene. The cells were kept under selection for 19 days. After 19 days, the cells were taken off selection and expanded for analysis. A proportion of the PGCs was kept under 300 μg/ml for another 31 days demonstrating that the PGCs were functionally resistant to the antibiotic.

Referring to FIG. 8, for the plasmid control, the cx-neo plasmid DNA was linearized with NotI and then digested with EcoRI or BamHI to produce a fragment that is slightly smaller (5 kb) than the intact plasmid which is shown by the HindIII digestion. Internal fragments of approximately 2 kb of the cx-neo plasmid were released by digestion with StyI or NcoI. A larger internal fragment of approximately 2.6 kb was released by digestion with EcoRI and KpnI. Digestion of genomic DNA from the line of PGCs with EcoRI, BamHI and HindIII revealed bands that are larger than 6 kb illustrating that the cx-neo transgene was incorporated into the PGC genome. The internal fragments revealed in plasmid DNA following digestion with StyI, NcoI and EcoRI with KpnI were also present in genomic DNA from the PGCs indicating that the cx-neo transgene was integrated into the PGC genome without alteration. Using convential transgene construction techniques, additional elements can be incorporated such as regulatory elements, tissue specific promoters and exogenous DNA encoding proteins are examples. Monoclonal antibodies are preferred example of a protein encoded by exogenouse DNA and human monoclonals are preferred species thereof.

As noted above, the performance of genetic modifications in PGCs to produce transgenic animals has been demonstrated in only a very few species. Analogous genetic manipulations can be achieved in chicken PGCs by referring to those achieved using ES cells in mice. In mice, the separate use of homologous recombination followed by chromosome transfer to embryonic stem (mES) cells for the production of chimeric and transgenic offspring is well known. Powerful techniques of site-specific homologous recombination or gene targeting have been developed (see Thomas, K. R. and M. R. Capecchi, Cell 51: 503-512, 1987; review by Waldman, A. S., Crit. Rev. Oncol. Hematol. 12: 49-64, 1992). Insertion of cloned DNA (Jakobovits, A., Curr. Biol. 4: 761-763, 1994) and manipulation and selection of chromosome fragments by the Cre-loxP system techniques (see Smith, A. J. et al., Nat. Genet. 9:376-385, 1995; Ramirez-Solis, R. et al., Nature 378:720-724, 1995; U.S. Pat. Nos. 4,959,317; 6,130,364; 6,130,364; 6,091,001; 5,985,614) are available for the manipulation and transfer of genes into mES cells to produce stable genetic chimeras. Many such techniques that have proved useful in mammalian systems would be available to be applied to chicken PGCs if the necessary cultures were available.

The genome of primordial germ cells is generally believed to be in a quiescent state and therefore the chromatin may be in a highly condensed state. Extensive testing of conventional electroporation protocols suggest that special methods are needed to introduce genetic modifications into the genome of PGCs. As described below, the transgenes may be surrounded with insulator elements derived from the chicken β-globin locus to enhance expression. The inclusion of the β-globin insulator elements routinely produces clones that can be grown, analyzed and injected into recipient embryos.

The conventional promoters that are used to drive expression of antibiotic (e.g. neomycin, puromycin, hygromycin, his-D, blasticidin, zeocin, and gpt) resistance genes are expressed ubiquitously. Typically, the promoters are derived from “housekeeping” genes such as β-actin, CMV, or ubiquitin. While constitutive promoters are useful because they are typically expressed at high levels in all cells, they continue to be expressed in most if not all tissues throughout the life of the chicken. In general, expression should be limited to only the tissue and stage of development during which expression is required. For selection of primordial germ cells, the period during which expression is required is their residence in vitro when the antibiotic is present in the media. Once the cells have been inserted into the embryo, it is preferable to terminate expression of the selectable marker (i.e. the antibiotic resistance gene). To restrict expression of the antibiotic resistance genes, the “early response to neural induction” (ERNI) promoter is used. An ERNI is a gene that is selectively expressed during the early stages of development (e.g. Stage X (E-G&K)) and in culture, and therefore, this promoter is used to drive expression of antibiotic resistance genes to select PGCs carrying a genetic modification. Since ERNI is only expressed during the early stages of development, the genes that confer antibiotic resistance are not expressed in the mature animals.

### EXAMPLE 9

### Homogeneity of Long Term PGC Cell Cultures

To determine the homogeneity of PGC cultures after long-term culture, ES, EG, DT40 (chicken B cell line) and PGCs were stained with anti-CVH, an antibody against the chicken vasa homologue and the 1B3 antibody (Halfter, W., Schurer, B., Hasselhorn, H. M., Christ, B., Gimpel, E., and Epperlein, H. H., An ovomucin-like protein on the surface of migrating primordial germ cells of the chick and rat. Development 122, 915-23. 1996)). Expression of the CVH antibody is restricted to germ cells and therefore, the anti-CVH antibody is a reliable marker for them. The 1B3 antigen recognizes an ovomucin-like protein present on the surface of chicken PGCs during their migration and colonization of the gonad.

Cells were washed in CMF/2% FBS, fixed in 4% paraformaldehyde for 5 minutes and washed again. The cell aliquots to be stained for vasa were permeabilized with 0.1% TritonX-100 for 1-2 minutes. Primary antibody was added for 20 minutes, cells were washed twice and incubated with a secondary antibody (Alexa 488 anti-rabbit IgG for CVH and control and Alexa 488 anti-rabbit IgM for 1B3) for 15 minutes. As controls, aliquots of cells were stained only with second antibody. After an additional 2 washes the cells were prepared for FACS analysis.

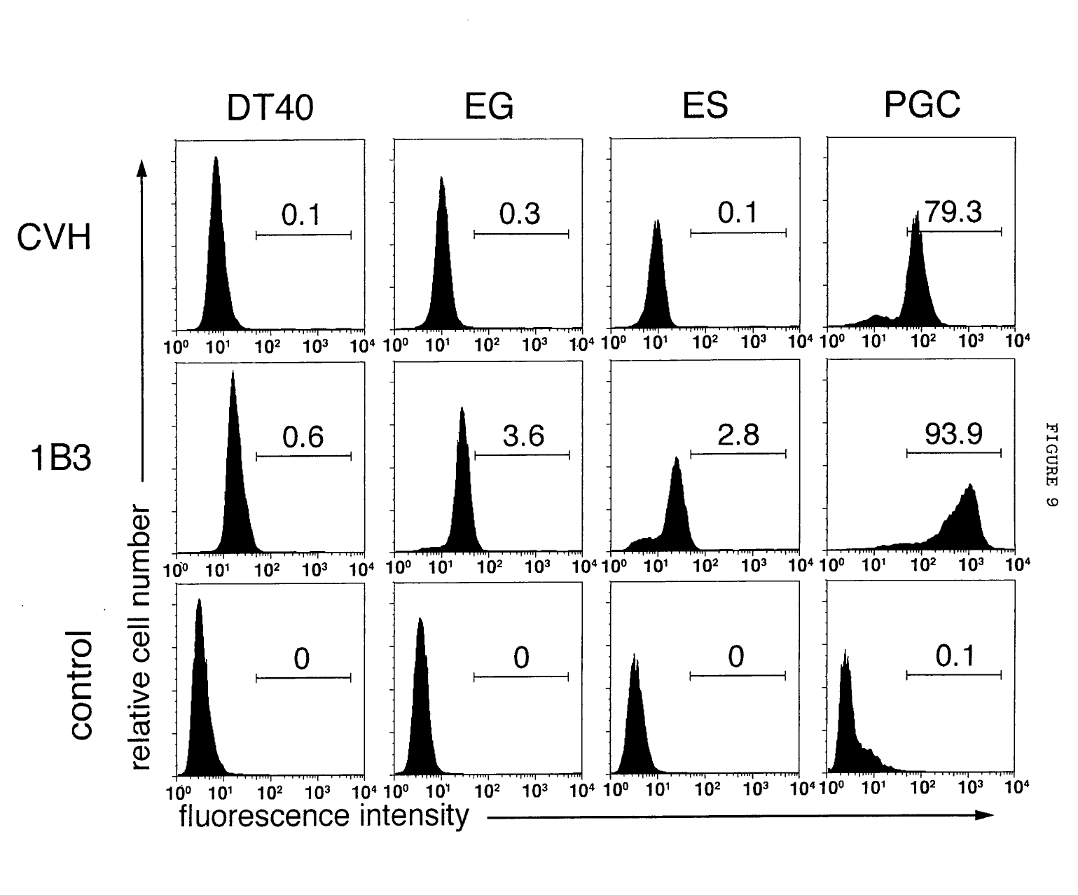

Referring to FIG. 9, DT40, ES and EG cells all show background when stained with CVH and the 1B3 Ab. PGCs, however, stain much stronger with both the CVH and the 1B3 antibody. There is a small population of PGCs, which do not stain for either CVH or 1B3 indicating that a small proportion of the cells do not display the PGC phenotype. Two parental PGC lines and 4 transfected cell lines (G-09, P84, P97/6 and P97/33) derived from the PGC13 parental cell line, were tested with the vasa and 1B3 antibody (PGC13 and 102). All show the same pattern indicating that the various PGC cultures contain the same high proportion of cells expressing the PGC phenotype.

### EXAMPLE 10

### Germline Transmission of Cultured PGCs

Primordial germ cell lines were derived from individual Barred Rock embryos. After establishment of the line, the cells were injected into Stage 13-15 (H&H) embryos. The embryos were grown to sexual maturity and the roosters were mated to Barred rock hens. Black offspring were indicative of germline transmission of the injected PGCs. All of the 21 males from the 4 different cell lines that have been tested for germline transmission to date have contributed to the germline. The minimum and maximum durations in culture were 35 days and 47 days, respectively (Table 2).

TABLE 2
Germline transmission of chimeric roosters

| | Cell | Days | # cells | % germline |
|---|---|---|---|---|
| Rooster | line | cultured | injected | transmission |
| IV14-32 | PGC21 | 44 | 1500 | 21 |
| IV14-41 | PGC21 | 44 | 1500 | 10 |
| IV14-47 | PGC21 | 44 | 1500 | 16 |
| IV15-03 | PGC35 | 35 | 3000 | 23 |
| IV15-04 | PGC35 | 35 | 3000 | 80 |
| IV15-12 | PGC35 | 35 | 3000 | 61 |
| IV15-18 | PGC35 | 35 | 3000 | 47 |
| IV15-21 | PGC35 | 35 | 3000 | 15 |
| IV15-27 | PGC35 | 35 | 3000 | 85 |
| IV15-33 | PGC35 | 35 | 3000 | 86 |
| IV16-02 | PGC34 | 47 | 3000 | 74 |
| IV16-03 | PGC34 | 47 | 3000 | 42 |
| IV16-13 | PGC34 | 47 | 3000 | 80 |

Of the PGC derived offspring a male and female were mated and their offspring incubated and hatched. A total of 7 eggs were set and all 7 hatched indicating that offspring derived from cultured PGCs can have normal reproductive function.

### EXAMPLE 11

### Genetic Modification of Primordial Germ Cells

Electroporation with a circular CX-GFP plasmid revealed that the rate of transient transfection in PGCs varied between 1-30%. Using 8 Square wave pulses of 100 μsec and 800V we obtained a PGC cell line carrying a CX-neo construct, that was designated G-09. See FIG. 8. The integration of the construct was evaluated using Southern blot analysis. The isolation of this stably transfected line, however, was a spurious event that did not recur in subsequent experiments. With the exception of G-09, stable transfection of PGCs was not achieved after electroporating 17×10⁷ PGCs with linearized constructs in 37 transfection experiments using both square wave and exponential decay pulses. In each of these experiments, the number of PGCs varied from 1×10⁶ to 10×10⁶. The following promoters, used widely in ES cell research in mouse, chicken and human were tested: the CX promoter, also called CAG (Niwa, H., Yamamura, K., and Miyazaki, J., Efficient selection for high-expression transfectants with a novel eukaryotic vector. Gene 108, 193-9.1991)), which contains the chicken β-actin promoter with a CMV enhancer, the PGK promoter, the MCI promoter and the Ubc promoter. None of these promoters increased transfection efficiency. To allow expression of selectable markers and clonal derivation of genetically modified cell lines, insulators have been used with integrated constructs.

Insulators are DNA sequences that separate active from inactive chromatin domains and insulate genes from the activating effects of nearby enhancers, or the silencing effects of nearby condensed chromatin. In chickens, the 5′HS4 insulator located 5′ of the β-globin locus has been well characterized by Felsenfeld and colleagues (Burgess-Beusse, B., Farrell, C., Gaszner, M., Litt, M., Mutskov, V., Recillas-Targa, F., Simpson, M., West, A., and Felsenfeld, G. (2002)). The insulation of genes from external enhancers and silencing chromatin. Proc Natl Acad Sci USA 99 Suppl 4, 16433-7. This insulator protects the β-globin locus from an upstream region of constitutively condensed chromatin. We assembled a transgene with the chicken β-actin promoter driving neomycin resistance using the chicken β-globin 5′HS4 sequence as insulators both 5′ and 3′ of the chicken β-actin-neo cassette.

The 250 bp core sequence of hypersensitive site 4 from the chicken β-globin locus was PCR amplified with the following primer set:

| | |
|---|---|
| HS4-Bam-F: | |
| AGGATCCGAAGCAGGCTTTCCTGGAAGG | (SEQ ID. NO. 7) |
| HS4-Bgl-R: | |
| AAGATCTTCAGCCTAAAGCTTTTTCCCCGT | (SEQ ID. NO. 8) |

The PCR product was cloned into pGEM-T and sequenced. A tandem duplication of the HS4 site was made by digesting the HS4 in the pGEM clone with BamHI and BglII to release the insert, and BglII to linearize the vector. The HS4 fragment was ligated to the vector containing a copy of the HS4 insulator, so that two copies were present. Clones were screened and one was selected in which the two copies of HS4 are in the same orientation. This is called 2×HS4.

### EXAMPLE 12

### Bulk Selection Using HS4 β-Acton-neo

β-actin neo was obtained from Buerstedde (clone 574) and transferred into pBluescript. 2×HS4 was then cloned at both the 5′ and 3′ ends of β-actin neo to produce HS4-β-actin neo. Eight transfections were performed using this construct. For each transfection 5×10⁶ PGCs were resuspended in 400 μl electroporation buffer (Specialty Media) and 20 μg of linearized DNA was added. A Exponential Decay (ED) pulse (200V, with 900-1100 μF) or a Square Wave (SW) pulses (250-350V, 8 pulses, 100 μsec) were given. After transfection the cells were grown for several days before neomycin selection (300 μg/ml) was added. Each transfection was grown as a pool. Resistant cells were isolated from 5 of 8 transfections

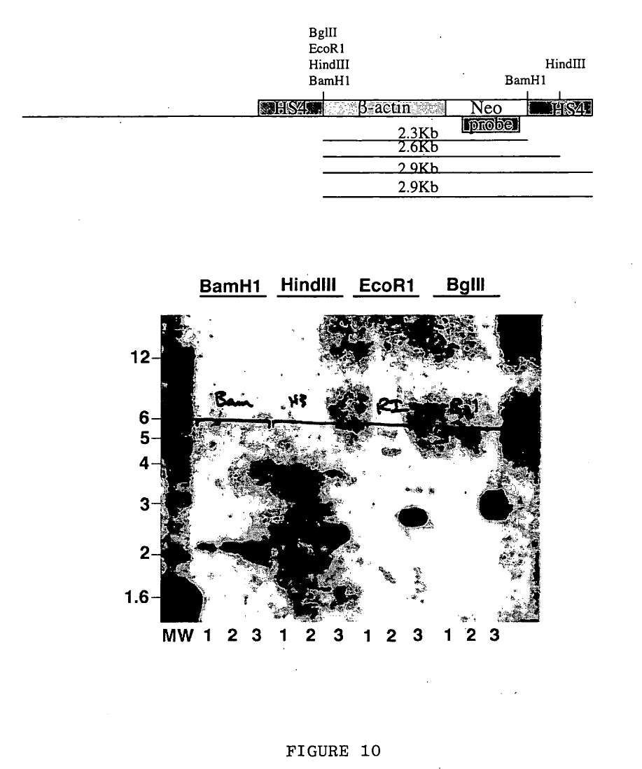

Southern analysis was performed on 2 pools of transfected cells (FIG. 10). Two μg genomic DNA from PGC lines P84 and P85 and 20 pg of plasmid (HS4-β-actin neo) were digested. Digests were run on a 0.7% gel, transferred by capillary transfer in 10×SSC to nylon membrane overnight, and probed with radiolabeled neo gene sequences for 2 hours in Rapid Hyb (Amersham). After washing, the blot was exposed to film overnight at −80° C. Referring to FIG. 10, Lane1 is P84, Lane 2 is P85 and Lane 3 is the plasmid. For the plasmid control the HS4-α-actin-neo plasmid DNA was linearized with Not1. To obtain a 2.3 Kb internal fragment the PGC DNA and the linearized plasmid were digested with BamH1. Both P84 and P85 show an internal fragment of 2.3 Kb in size. A larger internal fragment of approximately 2.6 Kb was released by digestion with HindIII. Again this internal fragment is present in both the P84 and P85 digests. Digestion of genomic DNA of P84 and P85 with EcoR1 and BglII should reveal bands larger than 2.9 Kb if the transgenes are integrated into the genome. In P84 no junction fragments are seen, indicating that P84 is a composite of several different clones. In P85, junction fragments of 4.5-5 kb are present in the EcoR1 digestion and a junction fragment of 5 Kb is present in the BglII digestion indicating that P85 is integrated into the genome and that the culture is comprised substantially from one clone. This example shows the utility of insulators as a preferred element of a construct for reliable expression of selectable markers in primordial germ cells.

### EXAMPLE 13

### Clonal Derivation of Genetically Modified PGCs

The following examples show that genetically modified lines of primordial germ cells can be clonally derived

First, β-actin-eGFP was made. The eGFP gene was released from CX-eGFP-CX-puro with XmnI and KpnI, β-actin was released from HS4-β-actin puro with EcoRI and XmnI, and the two were cloned as a 3-way ligation into pBluescript digested with EcoRI and KpnI to produce β-actin EGFP. Then, β-actin eGFP was released with BamHI and KpnI (blunted with T4 DNA polymerase) and cloned into HS4-β-actin puro digested with BglII and EcoRV.

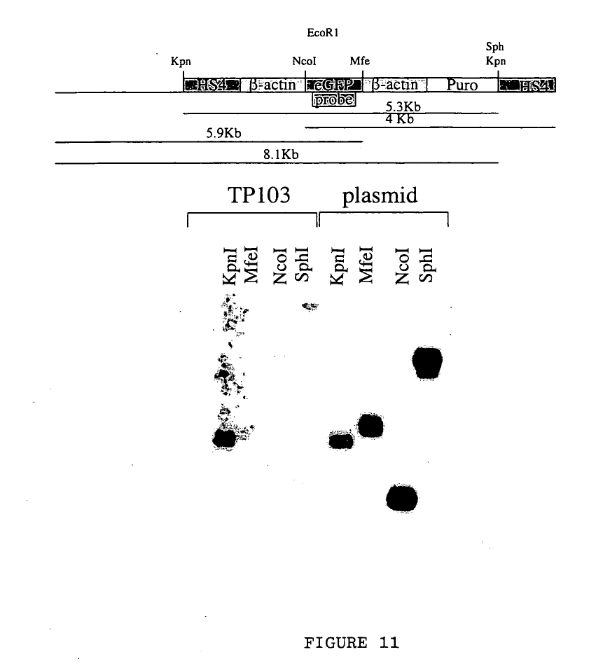

Five transfections were performed using this construct. For each transfection 5×10⁶ PGCs were resuspended in 400 μl electroporation buffer (Specialty Media) and 20 μg of linearized DNA was added. An ED pulse (150-200V; 900 μF) or SW (350V, 8 pulses, 100 μsec) pulses were given. After transfection the cells were plated into individual 48 wells and grown for several days before selection (0.5 μg/ml) was added. A total of 5 clones were observed in 4 of the 5 transfections. One clone TP103 was analyzed by Southern (FIG. 11). Referring to FIG. 11, the plasmid control DNA was linearized with NotI. An internal fragment was released by digesting the DNA with KpnI. In both TP103 and the plasmid a fragment of the same size was released. Digestion of genomic DNA of TP103 with NcoI, MfeI, and SphI should reveal bands that are larger than the corresponding lanes of digested plasmid DNA. No band is seen in the lane of MfeI digested TP103 genomic DNA, which may be due to the band being too large. In the lanes representing the NcoI and SphI digestions, fragments have been released in the TP103 genomic DNA that are substantially larger than the fragments released in the plasmid DNA, indicating that the transgene is incorporated into the genome of the TP103 cell line.

Clonal Derivation of HS4-β-actin-puro.

First, β-actin puro was made by a 3-way ligation of puro from CX-EGFP-CX-puro (XmnI-EcoRI), β-actin from β-actin neo in pBS (see above)(Sal-XmnI), and pBluescript (SalI-EcoRI). Next, β-actin puro was cloned into pBS containing two copies of 2×HS4 by ligating BamHI digested β-actin puro into BamHI/SAP treated 2×HS4 vector.

Three transfections were performed using this construct. For each transfection 4-5×10⁶ PGCs were resuspended in 400 μl electroporation buffer (Specialty Media) and 20 μg of linearized DNA was added. An ED pulse was given of 200V, 900 μF. After transfection the cells were plated into individual 48 wells and grown for several days before selection (0.5 μg/ml) was added. No colonies were seen in 2 transfections. Two colonies were isolated from the third transfection.

Clonal Derivation of HS4-cx-eGFP-cx-Puro.

Three transfections were performed with HS4-cx-eGFP-cx-Puro. 5×10⁶ PGCs were resuspended in 400 μl electroporation buffer (Specialty Media) and 20 μg of linearized DNA was added. Eight SW pulses of 350V for 100 μsec was given to each transfection. After transfection the cells were plated in individual 48 wells, grown for several days before puromycin selection (0.5 μg/ml) was added. A total of 16 clones were isolated from 2 transfections.

Clonal Derivation of cx-neo.

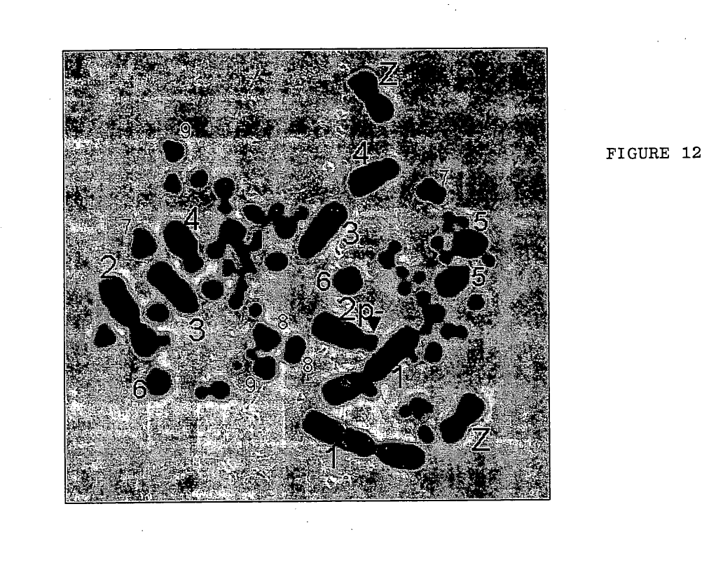

The PGC 13 cell line was electroporated with a plasmid carrying a cx-neo selectable marker. After exposure to neomycin a cell line was derived that was resistant to neomycin (G-09). The karyotype of this cell line was determined and all cells exhibited a deletion in the p-arm of chromosome 2 (Table 3 and FIG. 12). These data demonstrate that G-09 was clonally derived from a PGC carrying a signature deletion in the p-arm of chromosome 2.

TABLE 3
Chromosomal analysis of G-09 cell line.

| | Chromosomes | | | | | | |
|---|---|---|---|---|---|---|---|
| Cell | 1 | 2 | 2p- | 3 | 4 | Z | Micros |
| 1 | 2 | 1 | 1 | 2 | 2 | 2 | 69 |
| 2 | 2 | 1 | 1 | 2 | 2 | 2 | 44 |
| 3 | 2 | 1 | 1 | 2 | 2 | 2 | 56 |
| 4 | 2 | 1 | 1 | 2 | 2 | 2 | 56 |
| 5 | 2 | 1 | 1 | 2 | 2 | 2 | 65 |
| 6 | 2 | 1 | 1 | 2 | 2 | 2 | 67 |
| 7 | 2 | 1 | 1 | 2 | 2 | 2 | 59 |
| 8 | 1 | 0 | 1 | 1 | 2 | 1 | 38 |
| 9 | 2 | 1 | 1 | 2 | 2 | 2 | 65 |
| 10 | 2 | 0 | 1 | 2 | 2 | 2 | 55 |
| 11 | 2 | 1 | 1 | 2 | 2 | 2 | 43 |
| 12 | 2 | 1 | 1 | 2 | 2 | 2 | 59 |
| 13 | 2 | 0 | 1 | 2 | 2 | 2 | 55 |
| 14 | 2 | 0 | 1 | 2 | 2 | 2 | 33 |
| 15 | 1 | 1 | 1 | 2 | 2 | 2 | 56 |
| 16 | 2 | 1 | 1 | 2 | 2 | 2 | 62 |

### EXAMPLE 14

### Tissue Specific Expression of Selectable Markers in PGCs

The gene ERNI is expressed from the pre-primitive streak stage in the chicken embryo and is an early response gene to signals from Hensen's node Streit, A., Berliner, A. J., Papanayotou, C., Sirulnik, A., and Stem, C. D. (2000). Initiation of neural induction by FGF signalling before gastrulation. Nature 406, 74-8. Furthermore ERNI is expressed in chicken ES cells Acloque, H., Risson, V., Birot, A., Kunita, R., Pain, B., and Samarut, J. (2001). Identification of a new gene family specifically expressed in chicken embryonic stem cells and early embryo. Mech Dev 103, 79-91. The ERNI gene (also called cENS-1) has an unusual structure in which a single long open reading frame is flanked by a 486 bp direct repeat, in addition to unique 5′ and 3′ UTR sequences. Based on the idea that this structure is reminiscent of a retroviral LTR-like structure, Acloque et al. 2001 assayed different portions of the cDNA sequence for promoter/enhancer activity and found that a region of the unique sequence in the 3′ UTR acts as a promoter. PCR primers were designed essentially as described (Acloque et al., 2001) to amplify an 822 bp fragment of the 3′ UTR of the ERNI gene. After amplification of the ERNI sequences, they were cloned upstream of the neomycin-resistance gene, with an SV40 polyA site, to generate ERNI-neo (1.8 kb). The 2×HS4 insulator was then cloned on either side of the ERNI-neo selectable marker cassette.

Two transfections were performed with HS4-Erni-neo. 5×10⁶ PGCs were resuspended in 400 μl electroporation buffer (Specialty Media) and 20 μg of linearized DNA was added. In the first transfection a single ED pulse of 175V, 900 μF was given and in the second transfecton, 8 SW pulses of 100 μsec and 350V were given. After transfection the cells were plated in individual 48 wells, grown for several days before neomycin selection (300 μg/ml) was added. In the first transfection (ED pulse) 5 colonies were isolated, and in the second transfection (SW pulses) 11 colonies were isolated.

The isolation of stably transfected clones indicates that ERNI is expressed in PGCs and can be used as a tissue specific promoter.

### EXAMPLE 15

### Contribution of Transfected PGCs to the Germline

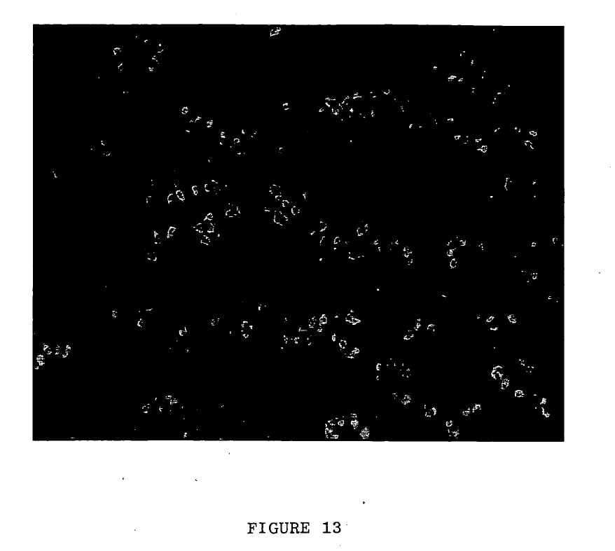

PGCs were transfected with HS4-βactin-GFP and injected into the vasculature of Stage 13-15 (H&H) embryos. At D18, gonads were retrieved, fixed, sectioned and stained with the CVH antibody to identify the germ cells. The stained sections were then analyzed for the presence of GFP positive cells in the gonads. GFP positive germ cells were found in both male (FIG. 13) and female gonads. Examination of histological preparations of brain, heart muscle and liver of these embryos showed only four green cells in one slide. These data demonstrate that a few cultured PGCs are found in ectopic locations but that the vast majority of cultured PGCs preferentially colonize the germline.

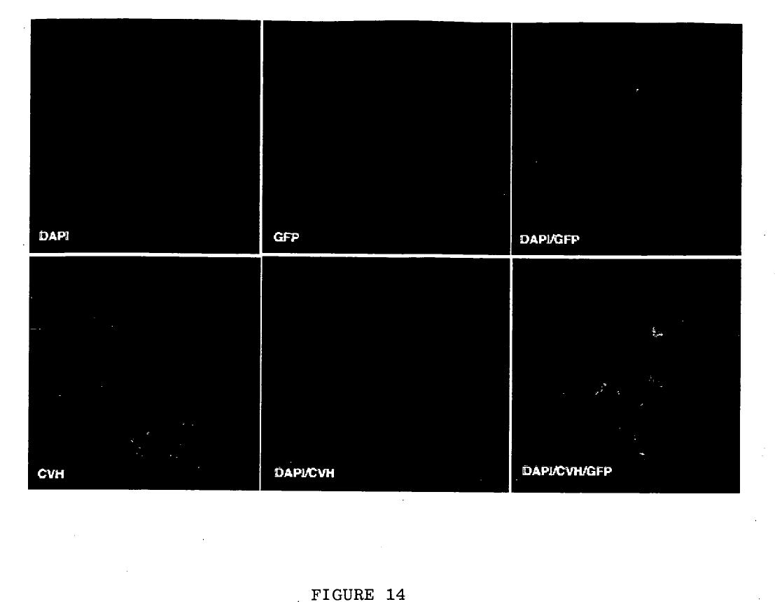

To determine that the GFP positive cells were germ cells the sections were stained with the anti-CVH antibody. As can be seen in FIG. 14, the GFP positive cells also stain for the CVH protein, indicating that the GFP positive cells are germ cells.

Referring to FIG. 14, GFP positive cells are present in this section and the DAPI/GFP panel shows that these GFP positive cells are located within the seminiferous tubule. When germ cells are stained with the anti-CVH antibody they exhibit a intense red stained ring that delineates the cytoplasm of the germ cells. The DAPI/CVH panel shows that these cells are located within the seminiferous tubule. The last panel shows that the GFP positive cells also stain for CVH and that the seminiferous tubules contains CVH positive germ cells that are GFP negative.

Although the examples herein are described for chickens, other gallinaceous species such as quails, turkey, pheasant, and others can be substituted for chickens under experimentation and with a reasonable expectation for successful implementation of the methods disclosed here.

We claim:

1. A clonally-derived cell culture of chicken primordial germ cells carrying exogenous DNA stably integrated genome of the primordial germ cells.

2. The culture of claim 1 wherein the culture medium is comprised of buffalo rat liver (BRL) cells.

3. The culture of claim 1 wherein the culture medium is comprised of fibroblast growth factor (FGF).

4. The culture of claim 1 wherein the culture medium is comprised of stem cell factor.

5. The culture of claim 1 wherein the culture medium is comprised of chicken serum.

6. The culture of claim 1 wherein the culture comprises at least 1×10⁵ cells.

7. The culture of claim 1 wherein the exogenous DNA is flanked by at least one insulator.

8. A germline chimeric chicken comprising:
germline tissues colonized by genetically modified primordial germ cells, and
somatic tissues substantially free of genetically modified cells.

9. The germline chimera of claim 8 wherein the genetic modification is the stable integration of exogenous DNA.

10. The germline chimera of claim 9 wherein the exogenous DNA encodes a protein.

11. The germline chimera of claim 10 wherein protein is a monoclonal antibody.

12. The method of claim 11 wherein the monoclonal antibody has a human polynucleotide sequence.

13. The germline chimera of claim 10 wherein the exogenous DNA is flanked by at least one insulator.

14. A primordial germ cell comprising a stably integrated transgene having an expressable early response to neural induction (ERNI) promoter operatively linked to a coding region of the transgene.


SEQUENCE LISTING (8 sequences)

SEQ ID NO: 1 - DNA, 20 bp, Artificial sequence V-1
  Primer to amplify a 751 bp fragment from CVH transcript.
gctcgatatgggttttggat

SEQ ID NO: 2 - DNA, 21 bp, Artificial Sequence V-2
  Primer to amplify a 751 bp fragment from CVH transcript.
ttctcttgggttccattctgc

SEQ ID NO: 3 - DNA, 20 bp, Artificial Sequence Dazl-1
  Primer to amplify a 536 bp fragment from Dazl transcript.
gcttgcatgcttttcctgct

SEQ ID NO: 4 - DNA, 19 bp, Artificial Sequence Dazl-2
  Primer to amplify a 536 bp fragment from Dazl transcript.
tgcgtcacaaagttaggca

SEQ ID NO: 5 - DNA, 21 bp, Artificial Sequence Act-RT-1
  Primer to amplify a 597 bp fragment from chicken actin transcript.
aacaccccagccatgtatgta

SEQ ID NO: 6 - DNA, 20 bp, Artificial Sequence Act-RT-2
  Primer to amplify a 597 bp fragment from chicken actintranscript.
tttcattgtgctaggtgcca

SEQ ID NO: 7 - DNA, 28 bp, Artificial Sequence  HS4-Bam-F
  PCR primer
aggatccgaagcaggctttcctggaagg

SEQ ID NO: 8 - DNA, 30 bp, Artificial Sequence HS4-Bgl-R
  PCR primer
aagatcttcagcctaaagctttttccccgt